(19) United States
(12) Patent Application Publication
(10) Pub. No.: US 2011/0313335 A1
(43) Pub. Date: Dec. 22, 2011

(54) THERAPY DEVICE AND METHODS OF USE THEREOF

(75) Inventors: AURELIA KOBY, Poway, CA (US); IAN MACMORRAN, Palm Desert, CA (US)

(73) Assignee: BROWN MEDICAL INDUSTRIES, SPIRIT LAKE, IA (US)

(21) Appl. No.: 12/818,424

(22) Filed: Jun. 18, 2010

(51) Int. Cl. A61F 5/00

(52) U.S. Cl. 602/4

(57) ABSTRACT

A therapy device for alleviating arm and shoulder pain and methods of use thereof is disclosed. In one embodiment, the therapy device is an arm sling with an adjustable, pressure-relieving neck strap. The arm sling may optionally include a squeezable pouch removably attached thereto. The squeezable pouch may be squeezed in the hand and/or manipulated by the fingers to either help relieve stress and muscle tension or to exercise the muscles of the hand.

### BACKGROUND OF THE INVENTION

Typical sling straps are of a woven material such as cotton, polyester, or nylon and tend to be of uniform width over the length of the strap. While the strap does have some flexibility, typically the strap is less flexible than the material comprising the envelope. At least one problem typically associated with the wearing of an arm sling is that the strap causes strain on the neck. In this respect, the wearing of an arm sling can be uncomfortable and cause neck pain to the wearer. A wearer who experiences uncomfortability and neck pain caused by the wearing of an arm sling may be prone to remove it prematurely resulting in a deceleration of the healing process.

### BRIEF SUMMARY OF THE INVENTION

A therapy device for alleviating arm and shoulder pain, including: (i) a fabric envelope having an open side end, a closed side end, a substantially open top and a closed bottom, the fabric envelope sized to retain a forearm arm of a person; (ii) a connecting strap attached to the fabric envelope at a corner where the open side end and the substantially open top meet; and (iii) a neck strap attached to the fabric envelope at a corner where the closed side end and the substantially open top meet, the neck strap including a first portion and a second portion wherein the second portion includes a plurality of pockets filled with a cushioning material is herein disclosed. The cushioning material may be a plurality of beads.

In one embodiment, the first portion of the neck strap includes a proximal end and a distal end and the second portion of the neck strap includes a proximal end and a distal end. The proximal end of the first portion may be attached to the fabric envelope and the distal end of the first portion may be attached to the proximal end of the second portion. The distal end of the second portion of the neck strap may include a ring attached thereto. The second portion of the neck strap may be an elastic material.

In one embodiment, the connecting strap includes a proximal end and a distal end. The proximal end may include fastening means integral thereto and the distal end may be attached to the fabric envelope at a corner where the open side end and the substantially open top meet. The proximal end of the connecting strap may be adapted to pass through the ring and the proximal end of the connecting strap may secure to the connecting strap at any portion there along. The fastening means includes a plurality of hooks of a hook-and-loop type fastener. In one embodiment, the connecting strap and the neck strap are secured to one another to form a continuous strap, the continuous strap being adjustable.

In one embodiment, the therapy device includes a squeezable pouch sized to substantially fit within a fist of the person and is removably attached to the fabric envelope at a corner where the open side end and the substantially open top meet. The squeezable pouch may include a cushioning material. The cushioning material may be a plurality of beads.

A therapy device for alleviating arm and shoulder pain, including: (i) means for retaining a forearm of a person; (ii) means for supporting the means for retaining the forearm about a neck of the person, the means for supporting having a plurality of housing means for housing a first set of cushioning material; and (iii) a squeezable pouch sized to substantially fit within a fist of the person and removably attached to the means for retaining the forearm, the squeezable pouch housing a second set of cushioning material is herein disclosed.

In one embodiment, the means for retaining the forearm is a fabric envelope, the fabric envelope including an open side end, a closed side end, a substantially open top and a closed bottom. In one embodiment, the housing means is a plurality of pockets adapted to rest against a back of the neck of the person. The first set of cushioning material and the second set of cushioning material may be a plurality of beads. The means for supporting may be adjustable. The means for supporting may include a supporting strap and an attaching strap, the supporting strap and the attaching strap adapted to removably connect.

### BRIEF DESCRIPTION OF THE DRAWINGS

FIG. 1 illustrates a perspective view of a therapy device according to an embodiment of the invention.

FIG. 2 illustrates a fabric envelope of the therapy device of FIG. 1 in an unsewn condition.

FIG. 3 illustrates a squeezable pouch detached from the therapy device of FIG. 1 according to an embodiment of the invention.

FIG. 4 illustrates a cross-sectional view of the squeezable pouch of FIG. 3.

FIG. 5 illustrates a bead which may be housed within the squeezable pouch of FIG. 3 or within a portion of a neck strap of the therapy device of FIG. 1 according to an embodiment of the invention.

FIG. 6 illustrates a neck strap of the therapy device of FIG. 1 according to an embodiment of the invention.

FIG. 7 illustrates a cross-sectional view of a pocket of a neck strap of the therapy device of FIG. 1 according to an embodiment of the invention.

FIG. 8 illustrates a person wearing the therapy device of FIG. 1.

### DETAILED DESCRIPTION OF THE PREFERRED EMBODIMENTS

The following detailed description is of the best currently contemplated modes of carrying out the present application. The description is not to be taken in a limiting sense, but is made merely for the purpose of illustrating the general principles of the present application.

Embodiments of the invention are directed to a therapy device for alleviating arm and shoulder pain and methods of use thereof. In one embodiment, the therapy device is an arm sling with an adjustable, pressure-relieving neck strap. The arm sling may optionally include a squeezable pouch removably attached thereto. The squeezable pouch may be squeezed in the hand and/or manipulated by the fingers to either help relieve stress and muscle tension or to exercise the muscles of the hand.

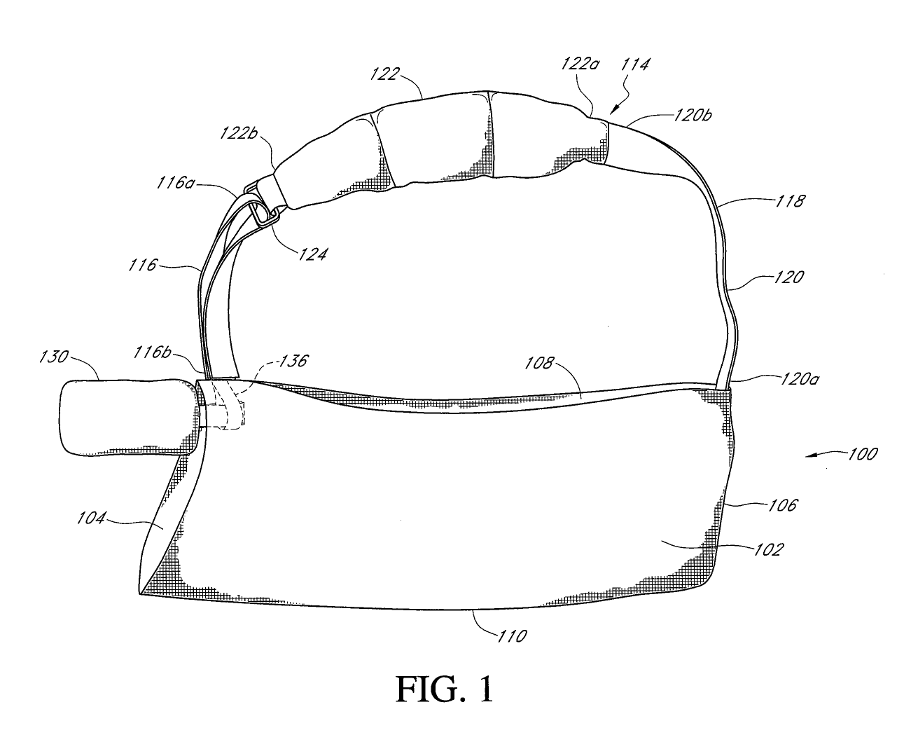

FIG. 1 illustrates a perspective view of a therapy device according to an embodiment of the invention. In one embodiment, therapy device 100 includes a fabric envelope 102 sized to retain an arm of a person. In some embodiments, fabric envelope 102 is approximately rectangular-shaped; however, other geometric shapes appropriate for application of the fabric envelope are within the scope of the invention. Fabric envelope 102 may include an open side end 104, a closed side end 106, a substantially open top 108 and a closed bottom 110. Fabric envelope 102 may be made of a stretchable fabric such as Lycra™, cotton-spandex, or equivalent materials, natural or synthetic. In any case, fabric envelope 102 may be made of a breathable fabric and may, for example, have a smooth interior and a textured exterior (such as mesh-like material). This construction may be desirable so that the sufficient air flow can reach the injured arm, especially in view of that the injured arm may be retained within therapy device 100 for an extended period of time. In some embodiments, fabric envelope may be coated with an anti-bacterial composition. This may substantially reduce bacterial growth that may proliferate, also in cases in which the injured arm is retained within therapy device 100 for an extended period of time.

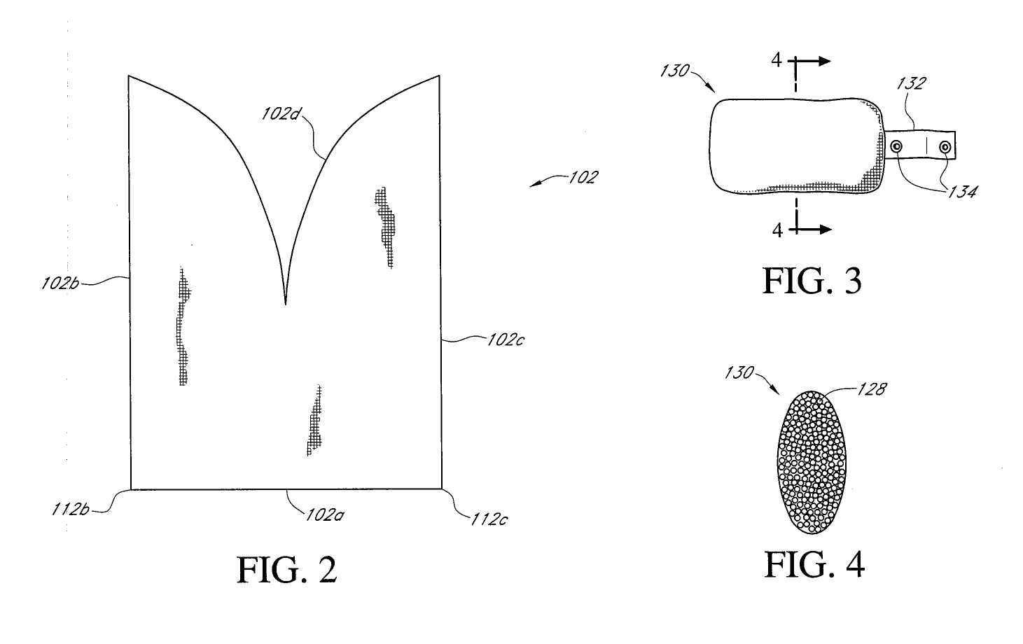

In some embodiments, fabric envelope 102 may be made of a unitary piece of fabric and sewn together at certain portions thereof to create fabric envelope 102. For example, in an unsewn condition, fabric envelope 102 may be approximately rectangular in shape with a substantial cut-out on at least one end. FIG. 2 illustrates fabric envelope 102 in an unsewn condition. Fabric envelope 102 (in an unsewn condition) includes side 102a, sides 102b and 102c (approximately parallel to one another), and side 102d with a substantial cut-out. In one embodiment, the substantial cut-out on side 102d approximate “tabs.” In one embodiment, fabric envelope 102 (in an unsewn condition) may be converted to fabric envelope 102 by bringing together sides 102b and 102c which may then be partially joined by sewing thereof beginning at corners 112b and 112c. As a result, open side end 104 of fabric envelope 102 may be accordingly formed. Meanwhile, the angled edges of side 102d (as shown) may be sewn together. As a result, substantially open top 108, closed side end 106 and closed bottom 110 of fabric envelope 102 may be accordingly formed. In other embodiments, fabric envelope 102 may be formed from a plurality of fabric panels sewn together.

Continuing to refer to FIG. 1, therapy device 100 includes a strap 114. In one embodiment, strap 114 includes a connecting strap 116 and a neck strap 118 forming a “continuous” strap 114. Connecting strap 116 includes a proximal end 116a and a distal end 116b. Connecting strap 116 may be attached to fabric envelope 102 at distal end 116b, i.e., at a corner where open side end 104 and substantially open top 108 meet, i.e., at corners 112b and 112c (see FIG. 2). The attachment may be effectuated by sewing or equivalent means. Proximal end 116a of connecting strap 116 may include a fastening means integral thereto. For example, the fastening means may be a plurality of hooks of a hook-and-loop type fastener or equivalent. In one embodiment, proximal end 116a of connecting strap 116 is capable of folding onto a portion of connecting strap 116 for securing thereof (explained in more detail below).

Neck strap 118 includes a first portion 120 and a second portion 122. First portion 120 may include a proximal end 120a and a distal end 120b. Similarly, second portion 122 may include a proximal end 122a and a distal end 122b. In one embodiment, proximal end 120a of first portion 120 is attached to fabric envelope 102 at an area in which closed side end 106 and substantially open top 108 meet, and distal end 120b of first portion 120 is attached to proximal end 122a of second portion 122. Each attachment may be effectuated by sewing or equivalent means. Distal end 122b of second portion 122 may include a securing means 124, such as a ring, attached thereto. In one embodiment, second portion 122 includes a plurality of pockets 126 filled with a cushioning material 128 (see FIGS. 6-7).

Continuing to refer to FIG. 1, therapy device 100 optionally includes a squeezable pouch 130. Pouch 130 may be made of an elastic fabric material. Pouch 130 may be sized to substantially position within a fist of a person and may be removably attached to fabric envelope 102 at the corner where open side end 104 and substantially open top 108 meet. In one embodiment, pouch 130 includes a tab 132 with a fastening means 134 which may be used to secure pouch 130 to therapy device 102 (see FIG. 3). Fastening means 134 may be, for example, a snap, button, hook, Velcro® or other type of fastener. In one embodiment, tab 132 includes a snap 134 which may pass through an elastic loop 136 (integral with therapy device 102) to secure pouch 130 thereon.

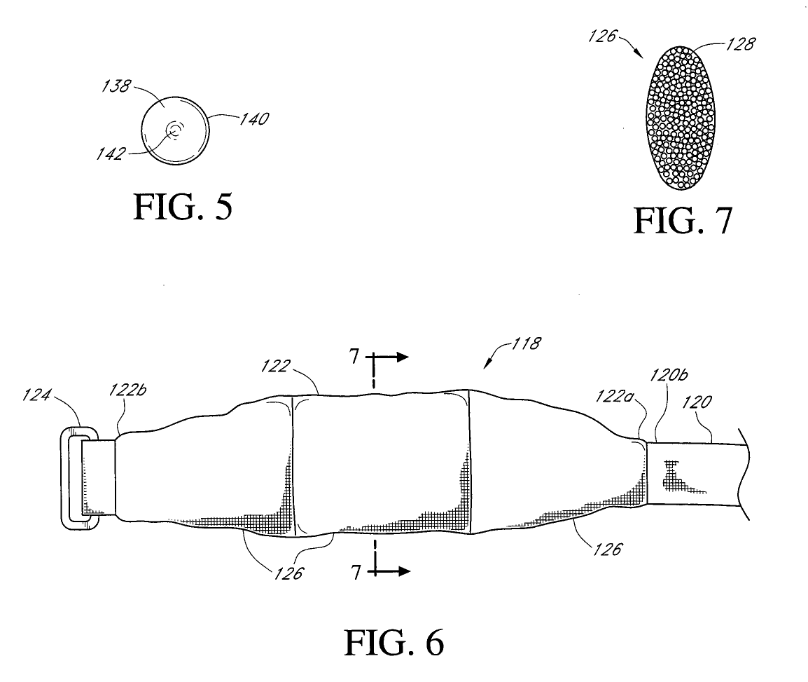

In one application, pouch 130 (see FIG. 3) may be squeezed in the hand and/or manipulated by the fingers to either help relieve stress and muscle tension or to exercise the muscles of the hand. Pouch 130 may be filled with cushioning material 128. FIG. 4 illustrates a cross-sectional view of pouch 130 of FIG. 3 taken along lines 4-4. In one embodiment, cushioning material 128 may be a granular filling material that is pellet or bead-shaped. The pellets or beads (hereinafter referred to as bead(s) 138, see FIG. 5) may be oval, elliptical, disc-shaped or round or any other similar geometric shape provided that the exterior is substantially or completely smooth. Cushioning material 128 may optionally include a lubricating material between and/or coating beads 138.

FIG. 5 illustrates a bead 138 according to an embodiment of the invention. Bead 138 includes exterior surface 140 and optionally may include one or more dimples such as dimple 142. In one embodiment, beads 138 may be formed of low density polyethylene (LDPE), and may be natural or semi-clear or white in color. Beads 138 may be injection molded or extrusion type LDPE particles. Beads 138 include little or no sharp edges and surface 140 is a smooth surface having an average surface roughness of less than one hundred (100) nanometers in one exemplary embodiment enabling beads 138 that form cushioning material 128 to slide easily and freely against and past each other. Surface 140 may be polished to enhance smoothness. A powdery coating may optionally be added to cushioning material 128, in particular on surfaces 140 of beads 138. The powdery coating may be a mold release agent formed of an amide or other materials, and which acts as a lubricant that promotes smooth sliding of the beads against one another.

FIG. 6 illustrates neck strap 118 of FIG. 1 in more detail. Second portion 122 of neck strap 118 may be made of an elastic fabric material. As shown, second portion 122 having proximal end 122a and distal end 122b includes pockets 126 filled with cushioning material 128 (see FIG. 7). In one embodiment, cushioning material 128 may be beads 138 (see FIG. 5) such as those described previously. Pockets 126 may number from one to about ten and may be separated from each other by, for example, sewing. Advantageously, neck strap 118 provides a cushioning effect to the neck of the wearer due to the cushioning material 128.

Also, the elasticity of the fabric material which includes at least second portion 122 of neck strap 118 allows for neck strap 118 to mold to the neck of a given wearer. In this respect, the wearer experiences less neck pain and is therefore less prone to remove therapy device 100 during the healing process. Also illustrated is proximal end 122a of second portion 122 connected to distal end 120b of first portion 120 of neck strap 118. FIG. 7 illustrates a cross-sectional view of a representative pocket 126 of second portion 122 of neck strap 118 taken along lines 7-7.

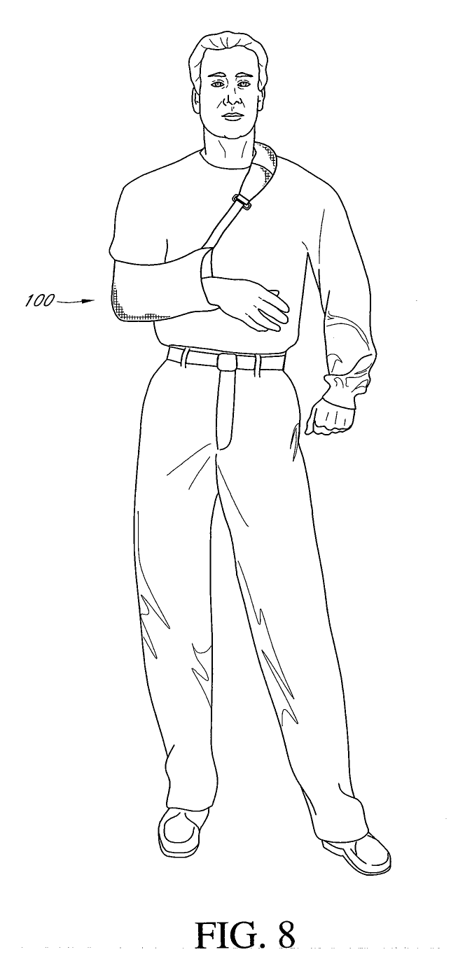

According to the embodiments of a therapy device as described, a forearm of a person can be positioned within fabric envelope 102 by placing the forearm and hand through open top 108 and passing the hand through open side end 104. Neck strap 118 can be brought around a neck of a person and connected to connecting strap 116 thereby suspending the forearm. For example, proximal end 116a of connecting strap may be passed through ring 124 (see FIG. 6) on distal end 122b of second portion 122 of neck strap 118 (see FIG. 6) to form a “continuous” strap 114 about the person's neck. Proximal end 116a having attaching means, such as Velcro®, can then be secured to connecting strap 116 at any portion there along. FIG. 8 illustrates a forearm of a person positioned within therapy device 100 according to an embodiment of the invention.

While certain exemplary embodiments have been described and shown in the accompanying drawings, it is to be understood that such embodiments are merely illustrative of and not restrictive on the broad application, and that this application is not be limited to the specific constructions and arrangements shown and described, since various other modifications may occur to those ordinarily skilled in the art.

What is claimed is:

1. A therapy device for alleviating arm and shoulder pain, comprising:
a fabric envelope having an open side end, a closed side end, a substantially open top and a closed bottom, the fabric envelope sized to retain a forearm arm of a person;
a connecting strap attached to the fabric envelope at a corner where the open side end and the substantially open top meet; and
a neck strap attached to the fabric envelope at a corner where the closed side end and the substantially open top meet, the neck strap comprising a first portion and a second portion wherein the second portion comprises a plurality of pockets filled with a cushioning material.

2. The therapy device of claim 1 wherein the first portion of the neck strap comprises a proximal end and a distal end and the second portion of the neck strap comprises a proximal end and a distal end, the proximal end of the first portion attached to the fabric envelope and the distal end of the first portion attached to the proximal end of the second portion.

3. The therapy device of claim 2 wherein the distal end of the second portion of the neck strap includes a ring attached thereto.

4. The therapy device of claim 3 wherein the connecting strap comprises a proximal end and a distal end, the proximal end comprising fastening means integral thereto, the distal end attached to the fabric envelope at a corner where the open side end and the substantially open top meet.

5. The therapy device of claim 4 wherein the proximal end of the connecting strap is adapted to pass through the ring, the proximal end of the connecting strap to secure to the connecting strap at any portion there along.

6. The therapy device of claim 4 wherein the fastening means comprises a plurality of hooks of a hook-and-loop type fastener.

7. The therapy device of claim 5 wherein the connecting strap and the neck strap are secured to one another to form a continuous strap, the continuous strap being adjustable.

8. The therapy device of claim 1 wherein the cushioning material is a plurality of beads.

9. The therapy device of claim 1 wherein the second portion of the neck strap comprises an elastic material.

10. The therapy device of claim 1, further comprising, a squeezable pouch sized to substantially fit within a fist of the person and removably attached to the fabric envelope at a corner where the open side end and the substantially open top meet.

11. The therapy device of claim 10 wherein the squeezable pouch includes a cushioning material.

12. The therapy device of claim 11 wherein the cushioning material is a plurality of beads.

13. A therapy device for alleviating arm and shoulder pain, comprising:
means for retaining a forearm of a person;
means for supporting the means for retaining the forearm about a neck of the person, the means for supporting having a plurality of housing means for housing a first set of cushioning material; and
a squeezable pouch sized to substantially fit within a fist of the person and removably attached to the means for retaining the forearm, the squeezable pouch housing a second set of cushioning material.

14. The therapy device of claim 13 wherein the means for retaining the forearm is a fabric envelope, the fabric envelope including an open side end, a closed side end, a substantially open top and a closed bottom.

15. The therapy device of claim 13 wherein the housing means is a plurality of pockets adapted to rest against a back of the neck of the person.

16. The therapy device of claim 15 wherein the first set of cushioning material and the second set of cushioning material comprise a plurality of beads.

17. The therapy device of claim 13 wherein the means for supporting is adjustable.

18. The therapy device of claim 13 wherein the means for supporting comprise a supporting strap and an attaching strap, the supporting strap and the attaching strap adapted to removably connect.
